SEQUENCE LISTING (5 sequences)

SEQ ID NO: 1 - protein, 5 aa, synthetic construct
  REGION 1..5: note = Peptide linker
GGGGS

SEQ ID NO: 2 - protein, 7 aa, synthetic construct
  REGION 1..7: note = Peptide linker
PKKKRKV

SEQ ID NO: 3 - protein, 22 aa, synthetic construct
  REGION 1..22: note = Peptide linker
SPVGGGGSRSPKKKRKVSPLEP

SEQ ID NO: 4 - protein, 17 aa, synthetic construct
  REGION 1..17: note = Peptide linker
SPVRSPKKKRKVSPLEP

SEQ ID NO: 5 - protein, 27 aa, synthetic construct
  REGION 1..27: note = Peptide linker
SPVGGGGSGGGGSRSPKKKRKVSPLEP

(19) United States
(12) Patent Application Publication
(10) Pub. No.: US 2025/0304935 A1
(43) Pub. Date: Oct. 2, 2025

(54) SPATIO-TEMPORAL CONTROL OF THE ACTIVITY OF A RNA-GUIDED DNA ENDONUCLEASE

(71) Applicants: Centre National de la Recherche Scientifique, PARIS (FR); Sorbonne Université, Paris (FR); Université Paris Cité, Paris 6 (FR); École Normale Supérieure, Paris (FR)

(72) Inventors: David BENSIMON, PARIS (FR); Bertrand DUCOS, GENTILLY (FR); Weiting ZHANG, BOSTON, MA (US); Pierluigi SCERBO, Bures sur Yvettes (FR)

(21) Appl. No.: 18/863,198

(22) PCT Filed: May 4, 2023
(86) PCT No.: PCT/EP2023/061858

§ 371 Date: Nov. 5, 2024

(30) Foreign Application Priority Data
May 6, 2022 (EP) 22305681.3

(51) Int. Cl. C12N 9/22; C07K 14/72; C12N 13/00; C12N 15/11; C12N 15/90

(52) U.S. Cl. CPC C12N 9/226; C07K 14/721; C12N 13/00; C12N 15/11; C12N 15/902; C07K 2319/09; C12N 2310/20

(57) ABSTRACT

The invention relates to a chimeric protein comprising three covalently linked domains including a RNA-guided DNA endonuclease, a flexible peptide linker, and a switchable receptor binding domain. The endonuclease activity depends on the uncaging of a caged specific ligand of said switchable receptor binding domain. Methods for inducing nuclear translocation of the chimeric protein and inducing the modification of an endogenous gene in a eukaryotic cell are also disclosed.

### CROSS-REFERENCE TO RELATED APPLICATIONS

The present application is a filing under 35 U.S.C. 371 as the National Stage of International Application No. PCT/EP2023/061858, filed May 4, 2023, entitled “SPATIO-TEMPORAL CONTROL OF THE ACTIVITY OF A RNA-GUIDED DNA ENDONUCLEASE,” which claims priority to European Application No. 22305681.3 filed with the European Patent Office on May 6, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4692-19600 BNT241986USPC Sequences Listing.xml; created on Oct. 23, 2024; and having a file size of 6.91 KB.
The information in the Sequence Listing is incorporated herein in its entirety for all purposes.

### FIELD OF THE INVENTION

The present invention relates to a chimeric protein and uses thereof. In particular, methods allowing a spatio-temporal control of the activity and/or expression of a RNA-guided DNA endonuclease are provided. These methods are based on the use of photoactivable ligands.

### BACKGROUND OF THE INVENTION

The CRISPR/Cas9 system offers a unique opportunity for genome editing, which is important for both fundamental and translational studies, as well as for therapeutic approaches. Therefore, optimizing the activity and specificity of this system is considered beneficial for the whole scientific community.

An important goal of development is to dispose of an inducible expression system, that may be used “on will” by researchers, allowing a spatiotemporal control of the expression of RNA-guided endonucleases such as Cas9.

In vertebrates, transgenic CRISPR/Cas9 system for in vivo genome editing has been used in mouse and zebrafish in order to study both developmental, physiological and pathological mechanisms (Liu et al., 2019).

The use of transgenic zebrafish lines engineered with tissue specific-promoters driving Cas9 expression (or tissue-specific expression of Gal4 driving UAS: Cas9 expression) (Di Donato et al., 2016) enables a more precise gene function analysis in vivo. Nevertheless, these systems of inducible expression present limits:
1) obtention of tissue-specific promoter sequences requires significant work, including identification and cloning tasks;
2) obtention of transgenic lines expressing exogenous Cas9 is also time and energy-consuming;
3) this system can be implemented only in established genetic animal models (e.g. mice and fishes), thus precluding such approaches to other canonical (e.g. amphibians, birds) and non-canonical animal models (e.g. bilaterian invertebrates);
4) the integration of exogenous Cas9 sequence into the target genome, prior to genome editing event, is highly unfavorable for future translational approaches;
5) Another limit of the system is that constitutive expression of Cas9 may impact on genome stability through off-target effects (Alanis-Lobato et al., 2021).

This is why spatiotemporal control of protein activity by optogenetic approaches would allow a more precise analysis of gene function, as precise as single-cell resolution.

Several strategies have been proposed to photo-control Cas9 and dCas9 activity in vitro (Hemphill et al., 2015; Nihongaki et al., 2015; Polstein and Gersbach, 2015).

However, several limitations due to slow activation time, more or less complex protein engineering biochemistry, and stability of tools can make Cas9 activation difficult in vivo.

Chimeric proteins comprising the hormone-binding domain of the estrogen receptor (ERT) have been previously reported and are extensively used for inducible nucleus translocation. The tamoxifen inducible system is one of the best-characterized “reversible switch” models. In this system, ERT is used as a regulatory domain: without its ligand tamoxifen, the receptor is maintained under inactive form, in an inhibitory complex, comprising intracellular chaperones such as Hsp90, localized into the cytoplasm. In presence of tamoxifen, the truncated receptor ERT is released from its inhibitory complex and the chimeric protein moves to the nucleus and becomes functional.

This tamoxifen inducible system has been successfully used for modifying genes in mice, with a chimeric protein comprising ERT fused to a Cre recombinase (Feil et al., 1997).

An improved domain designated as ERT2 has been engineered, being specific of the ligand 4-hydroxy-tamoxifen. A chimeric protein comprising Cas9 enzyme fused to two ERT2 domains (iCas) has been shown to be inactive without 4-hydroxy-tamoxifen, but with high editing efficiency in presence of the ligand (Liu et al., 2016).

This system has been improved by the use of caged photoactivable compounds derived from tamoxifen. The optical induction of the chimeric protein allows a temporal control of the activity of the chimeric protein, but also a spatial control with the possibility to irradiate only one cell among others in a living organism.

The international application WO 2013/158268 describes photoactivable, caged molecules (I-(4,5-dimethoxy-2-nitrophenyl)ethyl (DMNPE)) that are used for specific optical release of tamoxifen derivatives into eukaryotic cells following irradiation.

Other photoactivable tamoxifen derivatives have been engineered such as caged cyclofen-OH, that is currently commercially available as “Actiflash”, distributed by the company IDYLLE (Sinha et al., 2010aa).

In order to spatially and temporally modify genes with a CRISPR/Cas system, two photoactivable chimeric proteins comprising a RNA-guided DNA endonuclease have been engineered by the inventors.

These photoactivable chimeric proteins allow a spatial and temporal control of the endonuclease activity, in particular they allow the modification of genes in a single cell of a living organism.

More precisely, in a zebrafish model, it is hereby shown that the use of these chimeric proteins can inactivate a cell-type specific gene in pigmented cells of only one eye.

### SUMMARY OF THE INVENTION

The present invention concerns a chimeric protein comprising three covalently linked domains:
a RNA-guided DNA endonuclease;
a flexible peptide linker; and
a switchable receptor binding domain,
the endonuclease activity depending on the uncaging of a caged specific ligand of said switchable receptor binding domain.

The present invention also concerns a single-strand or double-strand polynucleotide encoding a chimeric protein as described above.

Another object of the invention is a vector of expression adapted for a eukaryotic cell, comprising a double-strand polynucleotide as defined above, under the control of a suitable promoter.

Another object of the invention is a eukaryotic cell expressing, transiently or permanently, a chimeric protein as defined above.

Another object of the invention is a method for inducing nuclear translocation of the chimeric protein as defined above in a eukaryotic cell, comprising:
providing a eukaryotic cell that contains said chimeric protein,
contacting said eukaryotic cell with a caged specific ligand of the switchable receptor binding domain of said chimeric protein,
irradiating the eukaryotic cell with a wavelength of light for a period of time sufficient to uncage the caged ligand, that will bind to the switchable receptor binding domain and induce the nuclear translocation of the chimeric protein.

The present invention also concerns a kit adapted for the implementation of this method, comprising:
a chimeric protein as defined above, or a single-strand polynucleotide encoding it, or a vector of expression comprising a double-strand polynucleotide encoding it;
a caged ligand, specifically binding to the switchable receptor binding domain of said chimeric protein in its uncaged form; and
optionally, light sources.

Another object of the invention is a method for inducing the modification of an endogenous gene in a eukaryotic cell, comprising:
providing a eukaryotic cell that contains:
a chimeric protein as defined above, and
guide RNA(s) adapted to modify said endogenous gene,
contacting said eukaryotic cell with a caged specific ligand of the switchable receptor binding domain of the chimeric protein,
irradiating the eukaryotic cell with a wavelength of light for a period of time sufficient to uncage the caged ligand, that will bind to the switchable receptor binding domain and induce the nuclear translocation of the chimeric protein,
wherein once in the nucleus, the RNA-guided DNA endonuclease will act in conjunction with said guide RNA(s) for modifying said endogenous gene.

Finally, the present invention also relates to a kit adapted for the implementation of this method, comprising:
a chimeric protein as defined above, or a single-strand polynucleotide encoding it, or a vector of expression comprising a double-strand polynucleotide encoding it;
guide RNA(s) adapted for modifying at least one specific gene;
a caged ligand, specifically binding to the switchable receptor binding domain of said chimeric protein under its uncaged form; and
optionally, light sources.

### BRIEF DESCRIPTION OF THE FIGURES

FIGS. 1A-1E: Use of a chimeric protein OPTO-Cas9 allows temporal gene inactivation in a cyclofen dependent manner.

FIG. 1A) Schematic representation of OPTO-Cas9 protein. An optimized Cas9 protein for zebrafish (black square) is fused, through a synthetic 22 amino acid (aa) sequence (LR1, SEQ ID NO.3) carrying a glycine (G) rich sequence (SEQ ID NO.1) and a Nuclear Localization Sequence (NLS, in light grey, SEQ ID NO.2), to a ligand-inducible ERT sequence (grey square).

FIG. 1B) In absence of active Cyclofen (caged cyclofen, cCyc, square), OPTO-Cas9 is sequestered by chaperons (CC, grey oval). Upon UV illumination (365-405 nm), cyclofen (Cyc) is released (uncaged) (circle) and, by binding ERT, can free OPTO-Cas9 from its inhibitory complex with cytoplasmic chaperones, thus allowing OPTO-Cas9 activation.

FIG. 1C) Transgenic zebrafish expressing EOSfp fluorescent protein were injected with 50 pg of OPTO-Cas9 mRNA plus 25 pg of gEOSfp at 1-cell stage and incubated with 3/μM of Cyc at 4 hpf (hpf: hours post-fertilization). Non-injected embryos were used as control (Ctrl). EOSfp fluorescence has been analysed in zebrafish at 2 dpf (dpf: day post-fertilization). Note the heterogeneous and reduced EOSfp fluorescence in embryos where OPTO-Cas9 has been activated by Cyc (n=15/16), when compared to the EOSfp fluorescence in control (ctrl) embryos (n=19/19). Quantification of EOSfp fluorescence indicates 40% reduction in EOSfp fluorescence by OPTO-Cas9 in zebrafish embryos (AU: Arbitrary Units).

FIG. 1D) Transgenic zebrafish expressing the fluorescent protein EOSfp (EOS Fluorescent Protein, were injected as in (A) and incubated with 6 μM of caged-cyclofen (cCyc) at 6-to-8 hpf. Global illumination (by UV 365 nm) for cCyc uncaging and OPTO-Cas9 activation was done at 9 hpf, for the embryo shown on the right of the figure. Note that injected embryos not incubated with cCyc were used as controls (Ctrl inj., on the left) in order to evaluate specificity and potential leakage (i.e., undesired activation) of OPTO-Cas9. EOSFP fluorescence has been analyzed in zebrafish embryos at 2 dpf. Note the heterogeneous and reduced fluorescence of EOSFPfp in embryos where OPTO-Cas9 has been activated by uncaging of cCyc (+UV 365 nm for 4 minutes), when compared to the EOSFP fluorescence in control (Ctrl inj.) embryos.

FIG. 1E) Schematic representation of the experimental approach for local activation of OPTO-Cas9 in zebrafish embryos in order to inactivate a gene in small group of cells or in a cell type of interest. mRNA coding for OPTO-Cas9 was injected in zebrafish embryos at 1-cell stage; the embryo is then incubated with cCyc at 4 hpf. Embryos were washed at 5 hpf. UV-mediated activation of OPTO-Cas9, by local uncaging of cCyc in a small group of cells, was done during epiboly (5-to-8 hpf) and morphological analyses were performed at larva stage (5 dpf).

FIGS. 2A-2B: OPTO-Cas9 allows precise spatiotemporal inactivation of the tyrosinase (tyr) gene and specific pigmentation defects in a cCyc+UV dependent manner.

FIG. 2A) Zebrafish wild-type embryos were injected with OPTO-Cas9 mRNA (together with gRNA against tyr) at 1-cell stage and incubated with 6 μM of cCyc at 4 hpf. Global UV illumination at 8 hpf (at 365 nm for 4 minutes) uncaged cCyc and activated OPTO-Cas9. Non-injected embryos were used as control (Ctrl). Pigmentation defects were analyzed in zebrafish at 2 dpf. Quantification of pigmentation defects indicates that 26.5% of embryos are of Class I phenotype (light grey), while 73.5% (55.1% Class II (medium grey)+18.4% Class III (black)) of embryos (n=49), where OPTO-Cas9 has been globally activated by cCyc (+UV for 4 min), displayed a widespread depigmentation phenotype as compared to control embryos (with 100% of Class I phenotype) (n=22).

FIG. 2B) Transgenic zebrafish Tg (ef1a: IoxP-GFP-STOP-IoxP-dsRED) were injected with OPTO-Cas9 mRNA (together with gRNA against tyr and CRE-ERT mRNA) at 1-cell stage and incubated with 6/μM of cCyc soon after injection. Local illumination (by UV 405 nm for 7 min) for cCyc uncaging and OPTO-Cas9 activation was done in a small group of presumptive RPE (Retinal Pigment Epithelium) cell progenitors of the ectoderm at 8 hpf, as shown in the schema. Following illumination and release of Cyc, CRE-ERT specifically floxed the exogenous GFP gene, allowing dsRED expression (and red fluorescence) in cells where Cyc was active, thus labelling cells where OPTO-Cas9 was activated. RPE defects and fluorescence has been analysed in zebrafish at 2 dpf. Note the strong and precise reduction of pigmentation in one eye (in brightfield, BF) of zebrafish embryo (also positive for dsRED fluorescence) (7/10) whereas the contralateral (non-illuminated) eye show neither pigmentation defects nor CRE-ERT mediated dsRED expression. Magnification of zebrafish head (on the right) show the precise co-localization of dsRED fluorescence within RPE cells in only one depigmented eye.

FIGS. 3A-3D: OPTO-Cas9 mediated spatiotemporal vox inactivation impacts on posterior axis (tail) formation in a cCyc+UV dependent manner.

FIG. 3A) Zebrafish nacre embryos were injected with OPTO-Cas9 mRNA (together with gVOX and mRFP mRNA, used as tracer) at 1-cell stage and incubated with 6 μM of cCyc at 4 hpf (sphere stage). Global illumination at 5-to-8 hpf (by UV 365 nm for 4 minutes) and local illumination (by UV 405 nm for 7 minutes) led to cCyc uncaging and OPTO-Cas9 activation. Note that injected embryos not incubated with cCyc (but submitted to global illumination at 365 nm) were used as controls (Ctrl inj.). Embryos were imaged using mRFP fluorescence. Scale bar indicates 500 μm in length.

FIG. 3B) Magnification of embryos injected as in (A). Note that head development was impaired only by global activation of OPTO-Cas9, whereas posterior axis (white arrowhead) formation was impaired by both global and local (tail only) activation of OPTO-Cas9 activity. Scale bar indicates 200 μm in length

FIG. 3C) Quantification of antero-posterior anomalies (A/P abn., medium grey) or only posterior anomalies (P.Abn., black) issued by global versus local (tail only) activation of OPTO-Cas9 compared to normal morphology (light grey). The total number of embryos for each condition (Control, Control injected, Global activation and Local activation) is indicated above the graph.

FIG. 3D) Embryos injected as in (A) at larval stage. Embryos were imaged using mRFP fluorescence and brightfield (BF). Scale bar indicates 500 μm in length. Note that eyes (grey arrowhead) and pectoral fin (white and black arrowhead) develop normally in Ctrl inj. and Local Act. conditions, whereas they fail to develop in Global Act. conditions. Brace indicates trunk and lines indicate trunk somite, brace indicates the tail and lines indicate tail somite. Arrowhead highlights the Trunk-to-Tail transition zone, whereas tail tip is indicated by white and black arrows. Representative larvae are shown. Quantification of somite number. Compared to Ctrl (white bar, n=4) and Ctrl inj. (black bar, n=4), global OPTO-Cas9 activation (horizontal lines bar, n=5) reduces both trunk and tail somite number, whereas local (tail) OPTO-Cas9 activation (diagonal lines bar, n=4) only affects tail somite number. Experiments were done 4 independent times.

FIGS. 4A-4D: Molecular analysis of vox gene inactivation by OPTO-Cas9

FIG. 4A) Schematic representation of the vox gene (Ensemble vox-201 ENSDART00000167949.3) with the location of the four guides gVOX and two primers used to target and amplify vox gene, respectively. The set of primers (forward 1 and reverse) generates a 1024 bp amplicon from gDNA.

FIG. 4B) A T7 endonuclease assay is performed. T7 Endonuclease I (T7E1) recognizes and cleaves non-perfectly matched DNA. T7E1 assay reveals a 900 bp fragment in gDNA from globally activated zebrafish only, revealing that a mutation appeared in the gene under these conditions only. The 900 bp band was extracted from the gel and purified (blue rectangle).

FIG. 4C) Hypothetical sequences of the T7E1 products on globally activated zebrafish if the 900 bp fragment results mainly from the guide gVOX1 activity.

FIG. 4D) Differential PCR amplifications of WT gDNA and purified 900 bp fragment with two different forward primers (FW1 and FW2) and a common reverse primer. PCR on WT gDNA amplifies 1024 bp and 810 bp with the FW1 and FW2 respectively. From the 900 bp fragment isolated after T7E1 treatment, only the 810 bp amplicon is amplified with FW2. No amplification was obtained with FW1 (highlighted in the red rectangle), suggesting that the cleavage by T7E1 assay occurs from mutations located in the gVOX1 region.

FIGS. 5A-5C: Phenotypic comparison of zebrafish larvae after global or local vox gene inactivation by OPTO-Cas9.

Zebrafish nacre embryos were injected with OPTO-Cas9 mRNA (together with gVOX and mRFP mRNA, used as tracer) at 1-cell stage and incubated with 6 μM of cCyc at 4 hpf (sphere stage). Global illumination (by UV 365 nm for 4 minutes) and local illumination (by UV 405 nm for 7 minutes) for cCyc uncaging and OPTO-Cas9 activation was done at 5-to-8 hpf.

FIG. 5A) Control embryos were injected, treated with UV but not incubated with cCyc. Control injected larvae display normal phenotype and correct head, trunk and tail morphology.

FIG. 5B) Local photoactivation of OPTO-Cas9 in presumptive tailbud cells strongly perturbs posterior axis morphology and tail elongation, without impacting head and trunk morphogenesis.

FIG. 5C) Global photoactivation of OPTO-Cas9 strongly impacts larvae morphogenesis. Extreme phenotypes lack cranial structures and show abnormal eye, head and ventral structures. The A/P axis fail to correctly elongate and to develop. Scale bars indicate 500 μm and white arrowheads show the frontier between trunk and tail somites.

FIGS. 6A-6C: Test of two different fusion proteins Cas9-NLS-ERT and Cas9-LR2-ERT

FIG. 6A) The chimeric protein Cas9-NLS-ERT is schematically represented, with a peptide linker of sequence SEQ ID NO.4. 200 ng of the purified 456 bp amplicons of the EOSfp locus were submitted to T7 endonuclease I test. Results are shown in the picture of the agarose gel: the amplicon of 456 pb is split up in two portions of 312 and 144 pb in cases where a mutation has been inserted into the EOSfp gene, i.e., where the fusion protein has been active on the target gene. In the present case, a band of 312 pb is also detected in absence of cyclofen (and in presence of gRNA against EOSfp), suggesting a leakage of the fusion protein.

FIG. 6B) The chimeric protein Cas9-LR2-ERT is schematically represented, including a peptide linker of sequence SEQ ID NO.5. Activity of Cas9-LR2-ERT against EOSfp gene in transgenic fishline tg(ubi: EOSfp) is presented in the graph with (+) or without (−) sgRNA EOSfp; and with (+) or without (−) induction with cyclofen and irradiation (UV). First vertical bar “WT” represents the control zebrafish embryos, not injected (N=15); the second vertical bar represents zebrafish embryos, injected with Cas9-LR2-ERT, but not activated (N=11, 6 viable embryos); the third vertical bar represents zebrafish embryos, injected with Cas9-LR2-ERT, after activation of the fusion protein (N=13, 5 viable embryos). These results suggest that injection of Cas9-LR2-ERT is lethal, and that this fusion protein is active with sgRNA EOSfp even in absence of UV activation (“leakage”).

FIG. 6C) Activities of Cas9-LR1-ERT and Cas9-LR2-ERT against tyrosinase gene in wild type zebrafish are presented in the left graph and in pictures of embryos. Different phenotypes of embryos are: WT-like (black); mosaic depigmentation (horizontal lines); strong depigmentation (diagonal lines) and abnormal (white).

In the boxes on the right, number of embryos having a specific phenotype among the four referenced are indicated, as obtained with each chimeric protein Cas9-LR1-ERT and Cas9-LR2-ERT. The number of embryos having the phenotype (class I, II or III) is indicated over the total number of injected embryos for each fusion protein.

FIGS. 7A-7B: Use of a chimeric protein OPTO-Cas9-GR allows temporal gene inactivation in a dexamethasone dependent manner

FIG. 7A) Zebrafish wild-type embryos were injected with OPTO-Cas9-GR mRNA, together with gRNA against tyr, at 1-cell stage and incubated with 10/μM of dexamethasone at 6 hpf for one hour. Zebrafish were rinsed and incubated in fresh embryo medium until phenotype analysis. Non-activated embryos were used as control (Ctrl, n=18). Pigmentation defects were analysed in zebrafish at 2 dpf. Phenotypes are illustrated by pictures on the left of the figure. In the table, the number of embryos having the phenotype (class I, II or III) is indicated as a percentage over the total number of injected embryos.

FIG. 7B) Zebrafish wild type embryos were injected with OPTO-Cas9-GR mRNA, together with gRNA against vox, at 1-cell stage and incubated with 10 μM of dexamethasone at 4 hpf (sphere stage) for one hour. Zebrafish were rinsed and incubated in fresh embryo medium until phenotype analysis. Morphology of embryos was analysed at 3 dpf and compared to non-activated embryos exhibiting wild type phenotype.

Phenotypes are illustrated by pictures on the left of the figure. In the table, the number of embryos having the phenotype (Vox+, Vox++ or Vox+++) is indicated as a percentage over the total number of injected embryos.

### DETAILED DESCRIPTION OF EMBODIMENTS OF THE INVENTION

In a first aspect, the present invention concerns a chimeric protein comprising three covalently linked domains:
a RNA-guided DNA endonuclease,
a flexible peptide linker, and
a switchable receptor binding domain,
the endonuclease activity depending on the uncaging of a caged specific ligand of said switchable receptor binding domain.

A “chimeric protein” designates a protein comprising or consisting in different domains originating from different natural or recombinant proteins, covalently linked together with at least one peptide linker. More precisely, a “chimeric protein” designates any single polypeptide unit that comprises two distinct polypeptide domains, wherein the two domains are not naturally occurring within the same polypeptide unit. Typically, such chimeric proteins are made by expression of a cDNA construct.

In the present case, the chimeric protein comprises two functional domains:
A RNA-guided DNA endonuclease, defined as an endonuclease able to unwind and cleave DNA at specific target sites indicated by RNA guide molecules, and
A switchable receptor binding domain, defined as a ligand-binding domain from a receptor, able to switch between a “Off” and a “On” position in presence of a specific ligand.

These at least two functional domains are covalently linked with a peptide linker.

In a specific embodiment of the invention, the chimeric protein consists in the three covalently linked domains cited above.

### Rna-Guided DNA Endonuclease

In a specific embodiment of the invention, the RNA-guided DNA endonuclease is a dual RNA-guided DNA endonuclease.

The most famous dual RNA-guided DNA endonuclease is SpCas9 (CRISPR associated protein 9, formerly called Cas5, Csn1, or Csx12) that has been identified in Streptococcus pyogenes (Jinek et al., 2012). Following its discovery as a natural bacterial defense mechanism against phages, the editing system CRISPR/Cas9 has been engineered as a programmable tool to cleave any nucleic acid sequence, that is targeted by specific guide RNA(s). Since then, several endonucleases capable of being used in this editing system have been discovered or engineered.

According to a specific embodiment of the invention, the RNA-guided DNA endonuclease is chosen among the following enzymes: Cas9, Cas12a (formerly Cpf1), Cas13a, Cas13b, and variants thereof.

In the sense of the invention, “Cas9, Cas12a, Cas13a and Cas13b” designate the wild-type endonucleases originating from a bacterial species, and also their orthologs originating from other bacterial species.

In particular, in the present application, the term “Cas9” designates any Cas9 enzyme from any bacterial species. While the first discovered SpCas9 has been identified in Streptococcus pyogenes, there exist also SaCas9 (from Staphylococcus aureus), NmCas9 (from Neisseria meningitidis), CjCas9 (from Campylobacter jejuni), StCas9 (from Streptococcus thermophilus) and TdCas9 (from Treponema denticola). Advantageously, the coding sequence for SaCas9 is ˜1 kb shorter than SpCas9, therefore SaCas9 can be efficiently packaged into an adeno-associated virus. In the sense of the invention, “variants” designate natural and engineered variants of Cas endonucleases, for example non-cleaving versions of Cas9, and engineered variants with enhanced activity and/or modified compatibility.

Non-cleaving versions of the wild-type enzymes, designated as “DeadCas”, have been identified and are designated as dCas9, dCas13, etc.

Examples of variants of Cas endonucleases include engineered variants able to recognize different PAM sequences. A non-extensive list includes: D1135E variant, VQR variant, EQR variant, VRER variant, variants with non-NGG PAM sequences, xCas9, and SpCas9-NG.

### Switchable Receptor Binding Domain

The chimeric protein according to the invention includes a switchable receptor binding domain.

A Receptor Binding Domain (RBD) consists in the domain of the receptor that binds the ligand.

In the sense of the invention, the adjective “switchable” designates the ability of a molecule to be reversibly shifted between two (or more) stable states. The molecules may be shifted between the states in response to environmental stimuli, such as changes in pH, light, temperature, an electric current, microenvironment, or in the presence of ions and other ligands.

A “switchable receptor binding domain” designates a receptor binding domain that can be reversibly shifted between two states, in response to the addition (or liberation) of a specific ligand.

Indeed, binding of said ligand induces a change of conformation of the switchable receptor binding domain. The receptor binding domain (RBD) will thus present two different states, that may be defined as “On” and “Off”, or “active” and “inactive”, or “state 1” and “state 2”, depending on the presence or absence of said ligand; or in other cases, depending on the biological availability of said ligand.

In that case, the RBD is said to be switchable, a consequence of its ability to switch between states. This change of conformation may be a structural change, inherent to an alteration of the protein 3D structure, and/or be a modification of partners in the complex, 3D structures including said RBD.

The switchable RBD of the estrogen receptor (ER) has been widely studied and engineered to yield the ERT domain that responds specifically to the estrogen-like ligand tamoxifen.

The tamoxifen-ERT inducible system is one of the best-characterized “reversible switch” models. In this system, ERT is used as a regulatory domain: without its ligand tamoxifen, the receptor is inactive, forming an inhibitory complex, with cytoplasmic chaperones such as Hsp90. In presence of tamoxifen, the estrogen binding domain ERT is released from its complex with chaperones and the chimeric protein can diffuse to the nucleus and become functional.

Other switchable domains, such as ERT2 specific of the ligand 4-hydroxytamoxifen (4-OHT or 4-HT), have also been engineered.

An interesting ligand of ERT2 is the analogue 4-hydroxycyclofen (4-OHC) that has a similar affinity as 4-OHT to ERT2, but is easier to synthetize and presents a better photostability.

Recently, photoactivable ligands and corresponding RBD have been developed. These ligands are bound to a photolabile protecting group termed “caging group” or “molecular cage”. When illuminated at an appropriate wavelength, the light releases this caging group and restores the native biological activity of the ligand. In consequence, said ligand exists under two forms designated as “caged” and “uncaged”. Photo-activation at a specific wavelength drives the “uncaging” of the caged ligand, thus releasing its active form, and allows its binding to the RBD.

The term “uncaging” literally means “to release from a cage”. In molecular biology, this term is used to designate the action of liberating a molecule whose activity is inhibited, in order to release an active molecule.

This term is mostly used for the photo-release of molecules from photolabile biologically inactive precursors (so-called caged compounds). When caged, the molecule is rendered biologically inactive by covalent attachment of a photochemically removable protecting group (“caging group” or “molecular cage”) to the key pharmacophoric functionality. Flash photolysis using light of a specific color cleaves the modifying group, i.e., triggers the uncaging, i.e., the photo-release of the molecule under its biologically active form. Advantageously, uncaging does not require any reagent, just light.

A “photoactivable ligand” means, in the present invention, a caged ligand. This ligand can be triggered by a flash of light. These molecules can be photochemically converted into active ligands (able to bind a specific receptor) when excited at the appropriate wavelengths.

The person of the art knows different photoactivatable ligands such as, for example, caged cyclofen, caged 4-hydroxycyclofen, caged tamoxifen and caged dexamethasone (See WO 2013/158268, (Sinha et al., 2010a) and (Hayashi et al., 2006)). Caged ligands are designated with a “c” in front of the abbreviation, for example: cCYC, cDex.

ERT antagonists such as tamoxifen and its derivatives can be caged and photo-released to control any chimeric protein comprising an ERT domain.

Caged tamoxifen derivatives have been described, for example, in the international application WO 2013/158268.

In particular, caged 4-hydroxycyclofen is currently commercially available as “Actiflash”, distributed by the company IDYLLE (Sinha et al., 2010a). Among the caged 4-hydroxycyclofen compounds, at least two versions are proposed: a caged cyclofen-OH that is photo-activated at a wavelength of about 365 nm (between 350 and 410 nm), and another one that is photo-activated at a wavelength of about 480 nm.

Another well-known switchable RBD is the hormone binding domain of the glucocorticoid receptor (GR), whose natural ligand is dexamethasone.

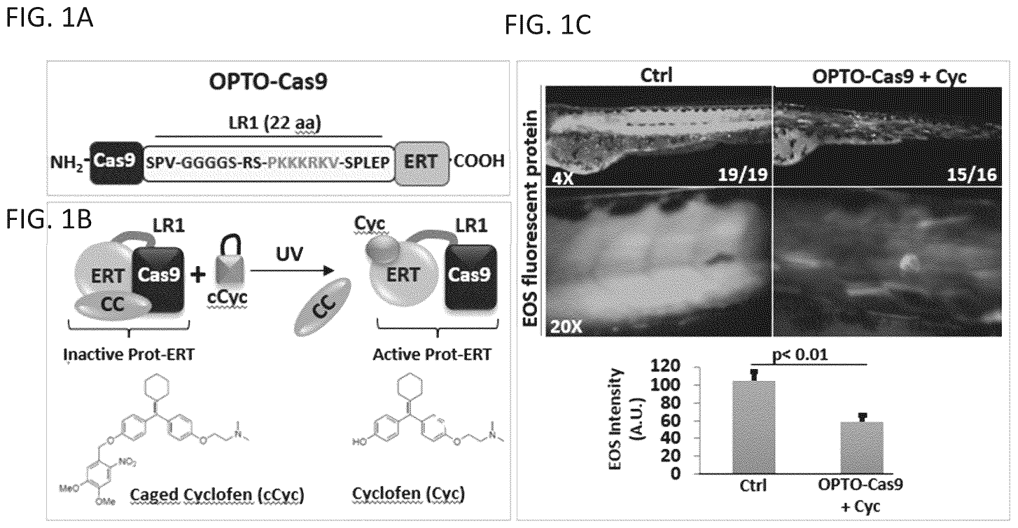

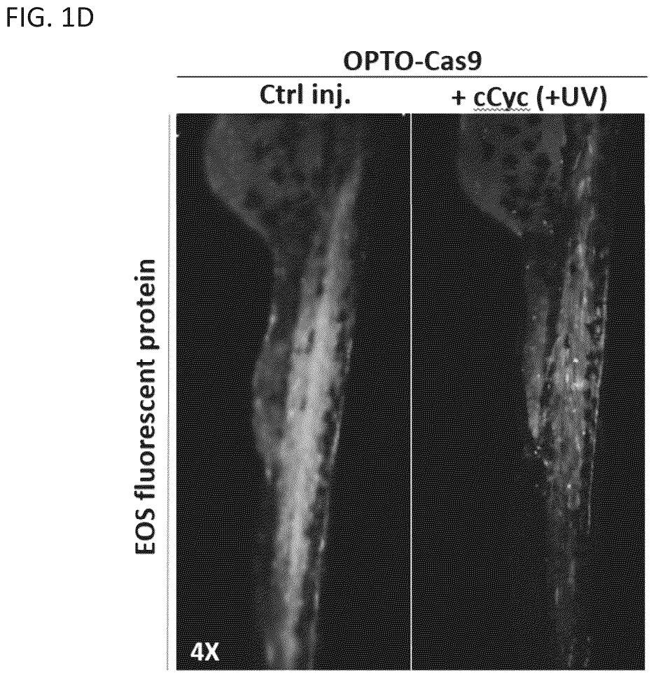

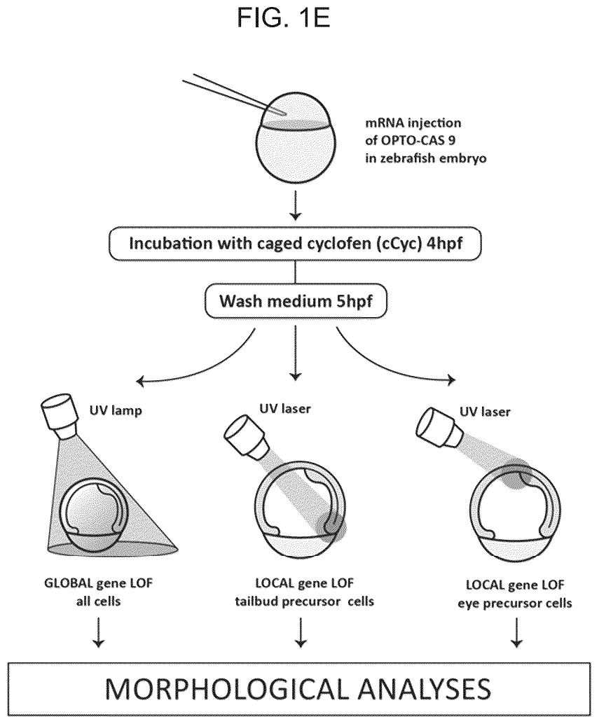

As described in (Hayashi et al., 2006), two new types of caged ligands, caged beta-estradiol and caged dexamethasone (cDEX), have been synthesized and successfully used in transgenic Arabidopsis plants carrying a GR-inducible transactivation system. According to a specific embodiment of the invention, in the chimeric protein, the switchable receptor binding domain is the hormone binding domain of the estrogen receptor (ER, ERT or ERT2) and the caged specific ligand is selected from caged cyclofen (cCYC, see formula in FIG. 1B), caged 4-hydroxycyclofen and caged tamoxifen. According to another specific embodiment of the invention, in the chimeric protein, the switchable receptor binding domain is the hormone binding domain of the glucocorticoid receptor (GR) and the caged specific ligand is a caged dexamethasone (cDEX).

In the sense of the invention, the sentence “the endonuclease activity depends on the uncaging of a caged ligand” means that the endonuclease activity depends on irradiation of a photoactivable ligand in its caged form driving the release (activation) of the ligand.

### Flexible Peptide Linker

Construction of chimeric proteins with specific functionality is a complex task that requires time and many attempts.

In particular, the nature and size of the peptide linker needs to be chosen carefully in order to achieve a functional chimeric protein.

Several types of peptide linkers have been referenced, including flexible linkers, rigid linkers and in vivo cleavable linkers.

Flexible linkers are useful when the joined domains require a certain degree of movement or interaction. They are composed of small amino acids (Ser, Gly, Thr, Ala, Lys and Glu), which provide flexibility between the joined domains. The most commonly used flexible linkers have sequences consisting in Gly and Ser residues.

The length of the peptide linkers is optimized to achieve appropriate separation of the functional domains and/or to maintain necessary inter-domains interactions.

In a specific embodiment of the invention, the chimeric protein comprises a flexible peptide linker that comprises between 17 and 27 amino acids.

The peptide linker may comprise between 18 and 26 amino acids, or between 19 and 25 amino acids, or between 20 and 24 amino acids, or between 21 and 23 amino acids. In a preferred embodiment of the invention, the peptide linker of the chimeric protein comprises 22 amino acids.

In another preferred embodiment of the invention, the flexible peptide linker of the chimeric protein comprises at least one of the following domains:
a flexible linker having one glycine (Gly) rich sequence, for example having a sequence GGGGS (SEQ ID NO. 1), and
a nuclear localization signal, for example having a sequence PKKKRKV (SEQ ID NO.2). Preferentially, the flexible peptide linker comprises both a glycine (Gly) rich sequence and a nuclear localization signal. More preferentially, the peptide linker of the chimeric protein of the invention comprises only one glycine rich sequence of SEQ ID NO. 1.

In a more preferred embodiment of the invention, the flexible peptide linker comprises the sequence SEQ ID NO. 3 (SPVGGGGSRSPKKKRKVSPLEP), in particular its sequence consists in SEQ ID NO. 3.

The chimeric protein according to the invention is also characterized by the fact that the endonuclease activity depends on the uncaging of a caged ligand specific of said switchable receptor binding domain. It is understood that in the sentence “uncaging of a caged specific ligand”, said photoactivable specific ligand is primarily under its caged form.

A “ligand specific of said switchable receptor binding domain” designates a ligand that binds with a significant affinity and specificity to the receptor binding domain contained in the chimeric protein. Another expression that is used in the present application, with the same meaning, is “a ligand specifically binding to the switchable receptor binding domain of said chimeric protein under its uncaged form, i.e., after uncaging”.

### Polynucleotides, Vectors of Expression and Transformed Eukaryotic Cells

The present invention also related to a single-strand or double-strand polynucleotide encoding a chimeric protein as defined above. In particular, this polynucleotide is a DNA or a RNA sequence.

Another object of the invention is a vector of expression adapted for a eukaryotic cell, comprising a double-strand polynucleotide encoding a chimeric protein as defined above under control of a suitable promoter.

A vector of expression adapted for a eukaryotic cell may be a viral vector or a plasmid. A suitable promoter is for example the cytomegalovirus (CMV) promoter, the ubiquitin promoter, the beta-actine promoter, or the beta-globin promoter.

The vector could also contain a suitable promoter for in vitro transcription, for example one of the bacteriophage promoters: SP6, T3 or T7 promoter.

This vector will allow the expression of the chimeric protein into the eukaryotic cell that contains it. This expression may be permanent or transitory, according to the nature of the vector of expression and of the regulatory elements, in particular according to the nature of the promoter.

The present invention also relates to a eukaryotic cell expressing, transiently or permanently, a chimeric protein as defined above. This eukaryotic cell may be prepared by any technique well known by the person of the art. In particular, this eukaryotic cell contains a vector of expression as defined above.

This eukaryotic cell is a mammal cell or a non-mammal cell. As an example, this eukaryotic cell is a non-human mammal cell. As another example, this cell is a zebrafish cell.

### Methods of the Invention

The present invention also relates to a method for inducing nuclear translocation of the chimeric protein as defined above in a eukaryotic cell, comprising:
providing a eukaryotic cell that contains said chimeric protein,
contacting said eukaryotic cell with a caged specific ligand of the switchable receptor binding domain of the chimeric protein,
irradiating the eukaryotic cell with a wavelength of light for a period of time sufficient to uncage the caged specific ligand, that will bind to the switchable receptor binding domain and induce the nuclear translocation of the chimeric protein.

The terms “eukaryotic cell that contains said chimeric protein” mean that the concerned cell expresses the chimeric protein, or that said chimeric protein has been introduced into said cell, by any method known to the person of the art. In particular, a messenger RNA (mRNA) encoding this protein can be introduced into said cell, thus inducing the translation of the messenger RNA into said protein.

The terms “contacting said eukaryotic cell with a caged specific ligand” mean that the eukaryotic cell is put into contact with this caged specific ligand. The person of the art knows different technologies for obtaining this contact. For example, in vitro, cells are incubated in a medium supplemented with said caged ligand; in vivo, said caged ligand may be administered to an organism in a way to penetrate it and then to target the cells of interest.

The terms “irradiation with a wavelength of light” mean that the eukaryotic cell(s) are illuminated with light of a given wavelength.

Light is a flow of photons carried by an electromagnetic wave emitted by an energy source designated as a “light source” such as the sun, a lamp, a photodiode or a laser. This flow of photons is characterized by their wavelength, which is directly related to their energy. By property, the shorter the wavelength, the more energy it carries. Light wavelengths (A) have the unit nanometre (nm) as their unit. In this class are included ultraviolet (UV) from 200 to 400 nm, blue from 400 to 500 nm, green from 500 to 600 nm, red from 600 to 700 nm, near infrared (NIR) from 700 to 800 nm and finally infrared (IR) from 800 to 1200 nm.

Monochromatic radiation is a beam with only one wavelength. Polychromatic radiation is radiation that contains several wavelengths.

In the method of the invention, the light sources that might be used include lamps, photo-diodes and monochromatic laser sources.

In the method of the invention, the irradiation for uncaging may be performed in different conditions, in particular at different wavelengths.

The uncaging might be performed with a monophoton illumination or a multi-photon illumination, in particular a biphoton or triphoton illumination.

Preferred wavelength for uncaging is:
monophoton, comprised between 350 and 405 nm;
biphoton, preferably at 750 nm;
triphoton, preferably at 1050 nm.

The duration of irradiation depends on the nature of the caged ligand and of the nature of the applied wavelength. This period is usually inferior to 5 minutes with a UV monophoton light source. In a multi-photon illumination, the period is usually inferior to 30 seconds.

In a specific embodiment, irradiation with multiphoton light sources consists in pulses of about 200 femtoseconds.

Once the uncaged ligand binds to the switchable receptor binding domain, the chimeric protein is released from its inhibitory complex with cytoplasmic proteins. Said chimeric protein is then free to diffuse in the cell and in particular to translocate in the nucleus. In another aspect, the invention concerns a method for inducing the modification of an endogenous gene in a eukaryotic cell, comprising:
providing a eukaryotic cell that contains:
a chimeric protein as defined above, and
guide RNA(s) adapted to modify said endogenous gene,
contacting said eukaryotic cell with a caged specific ligand of the switchable binding domain of the chimeric protein,
irradiating the eukaryotic cell with a wavelength of light for a period of time sufficient to uncage the caged specific ligand, that will bind to the switchable receptor binding domain and induce the nuclear translocation of the chimeric protein, wherein once in the nucleus, the RNA-guided DNA endonuclease will act in conjunction with said guide RNA(s) for modifying said endogenous gene.

In a specific implementation of the methods described above, the caged ligand is chosen among the group consisting of: caged cyclofen, caged 4-hydroxycyclofen, caged dexamethasone, and caged tamoxifen.

Naturally, this ligand will be chosen according to the nature of the switchable RBD of the chimeric protein, in order to assure its specific binding to said RBD.

These methods are performed in vitro, ex vivo or in vivo in a living body.

When the methods described above are realized in vivo, in a living body, the caged specific ligand may be orally administered to the body, by any technique known to the person of the art.

### Kits for the Implementation of the Methods

The present invention also relates to kits for the implementation of the methods described above.

In a first aspect, the invention concerns a kit for the implementation of a method for inducing nuclear translocation of the chimeric protein as defined above in a eukaryotic cell, comprising:
a chimeric protein as described above, or a single-strand polynucleotide encoding said chimeric protein, or a vector of expression comprising a double-strand polynucleotide encoding said chimeric protein;
a caged specific ligand, specifically binding to the switchable receptor binding domain of said chimeric protein under its uncaged form, i.e., after uncaging; and
optionally, light sources.

In a second aspect, the invention concerns a kit for the implementation of a method for inducing the modification of an endogenous gene in a eukaryotic cell, comprising:
a chimeric protein as described above, or a single-strand polynucleotide encoding said chimeric protein, or a vector of expression comprising a double-strand polynucleotide encoding said chimeric protein;
guide RNA(s) adapted for modifying said endogenous gene;
a caged specific ligand, specifically binding to the switchable receptor binding domain of said chimeric protein under its uncaged form, i.e., after uncaging; and
optionally, light sources.

Light sources are for example manual UV lamps.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### Example 1. Construction of a Chimeric Protein “OPTO-Cas9” or “Cas9-LR1-ERT”

A chimeric protein OPTO-Cas9 was constructed, where an optimized Cas9 for expression in zebrafish (Jao et al., 2013) is fused to the ERT receptor with a flexible linker sporting an NLS (Nuclear Localization Sequence) and a glycin-rich sequence between the two proteins.

This construction, also designated as Cas9-LR1-ERT, is presented in FIG. 1A.

In this chimeric protein, the peptide linker is of sequence SEQ ID NO. 3. It comprises:
a flexible linker having a glycine rich sequence, GGGGS (SEQ ID NO. 1), and
a nuclear localization signal having a sequence PKKKRKV (SEQ ID NO.2).

This peptide linker has been optimized for obtaining an OPTO-Cas9 that is 100% inactive in absence of cyclofen, and becomes active after release of cyclofen in the cytoplasm of the cell.

Two other chimeric proteins Cas9-NLS-ERT and Cas9-LR2-ERT, with different peptide linkers, were tested:
the chimeric protein Cas9-NLS-ERT comprises a peptide linker of SEQ ID NO. 4: SPVRS-PKKKRKV-SPLEP, which does not contain any glycine rich sequence;
the chimeric protein Cas9-LR2-ERT comprises a peptide linker of SEQ ID NO. 5: SPV-GGGGSGGGGS-RS-PKKKRKV-SPLEP, which contains two glycine rich sequences (2×SEQ ID NO.1).

Cas9-NLS-ERT was first tested on EOSfp gene in the transgenic line tg(ubi: EOSfp) of zebrafish, ubiquitiously expressing EosFP marker protein. EosFP is a fluorescent marker protein with UV-inducible green-to-red fluorescence conversion. This protein emits strong green fluorescence (516 nm) that changes to red (581 nm) upon near-UV irradiation at ≈390 nm.

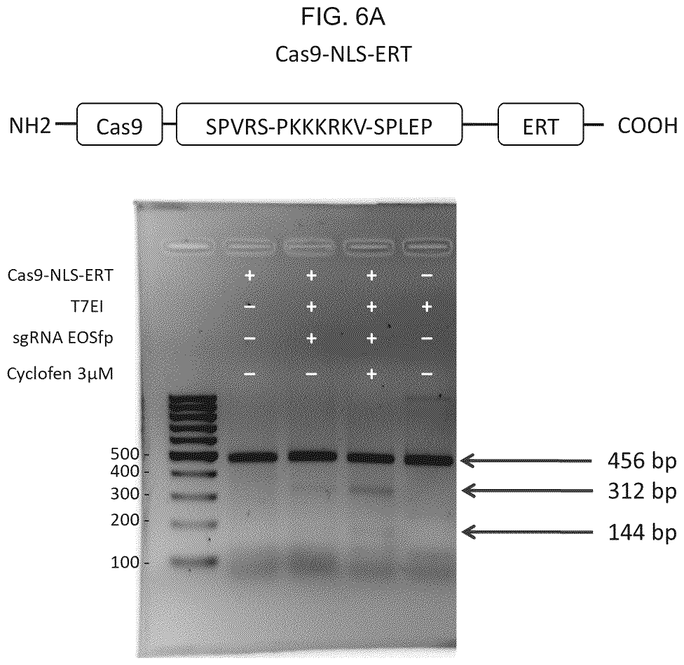

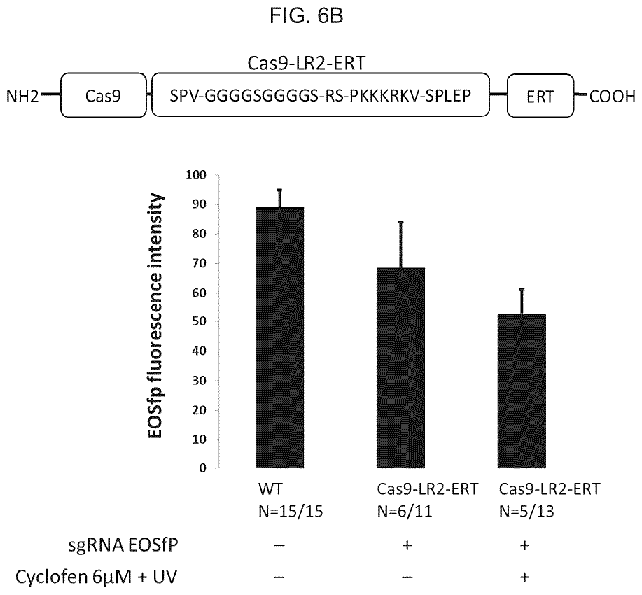

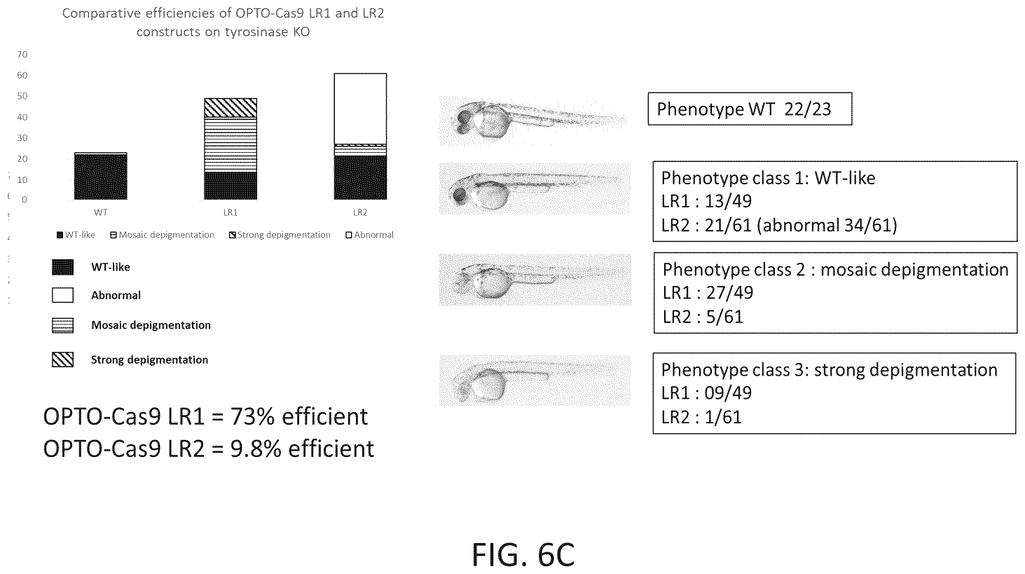

After injection of Cas9-NLS-ERT mRNA (150 pg) with sgRNA against EOSfp (25 pg) at one cell-stage, embryos were incubated with 3 μM regular cyclofen (at 521 cell stage) for two hours. Fish were fixed at 48 dpf (days post-fertilization) before gDNA extraction. 200 ng of the purified 456 bp amplicons of the EOSfp locus were submitted to T7 endonuclease I test. (FIG. 6A). Activity of Cas9-NLS-ERT plus sgRNA against EOSfp is present even in absence of cyclofen, indicating “leakage” of the chimeric protein. Activity of Cas9-LR2-ERT against EOSfp gene in transgenic fishline tg(ubi: EOSfp) is presented in FIG. 6B. Activity of Cas9-LR2-ERT against tyrosinase gene in wild type zebrafish is presented in FIG. 6C. It is apparent from these data that Cas9-LR2-ERT exhibits leakage and toxicity.

The subsequent experiments have all been performed with the chimeric protein OPTO-Cas9 (Cas9-LR1-ERT) presented in FIG. 1A.

In order to evaluate whether OPTO-Cas9 effectively responds to Cyc-mediated activation at desired time in already spatially organized embryonic cells, we first choose to inactivate an exogenous EOSfp fluorescent protein in a Tg (ubi: EOSfp) zebrafish.

Thus, we injected OPTO-Cas9 mRNA plus gEOSfp in Tg (ubi: EOSfp) zebrafish embryos at 1-cell stage and incubated with Cyc as later as 4 hpf (hours post-fertilization, sphere stage), when embryos counts≈4 000 embryonic cells

At 2 dpf (days post-fertilization), zebrafish embryos showed reduced EOSfp fluorescence when compared to control non-injected embryos (FIG. 1C).

Closer inspection of zebrafish somites and whole fluorescence quantification further confirm that OPTO-Cas9 reduces EOSfp fluorescence when activated at 4 hpf, indicating that activation of OPTO-Cas9 can be effective in a large field of embryonic cells as late as 4 hpf.

In order to evaluate whether OPTO-Cas9 can be effectively controlled by cCyc (caged cyclofen) with minimal leakage, we injected OPTO-Cas9 mRNA plus gEOSfp in Tg (ubi: EOSfp) zebrafish embryos at 1-cell stage, incubated with cCyc from 4 to 5 hpf (1 h of treatment) and shined with UV (2=365 nm) for 4 minutes. In parallel, injected embryos illuminated with UV but not incubated in cCyc were used as controls of leakage.

We observed that EOSfp fluorescence was exclusively reduced in embryos incubated with cCyc (activated at 9 hpf), but not in control injected and untreated embryos. This indicated that OPTO-Cas9 can be precisely activated at desired time point(s) in order to induce gene inactivation, with minimal leakage and/or unspecific effects.

The quality of OPTO-Cas9 mRNA after in vitro transcription, used in the following examples, has been verified before injection to embryos.

The mRNA was analyzed by capillary electrophoresis, which revealed as expected a unique mRNA band at =6 kb with an RNA Quality Number equal to 9.4 (on a scale ranging from 0 to 10), indicating the high quality of the preparation.

### Example 2. Use of OPTO-Cas9 to Inactivate a Cell-Type Specific Gene in One Cell

We further characterized the chimeric protein OPTO-Cas9 by analysing its capacity to inactivate endogenous genes in a spatiotemporal defined manner.

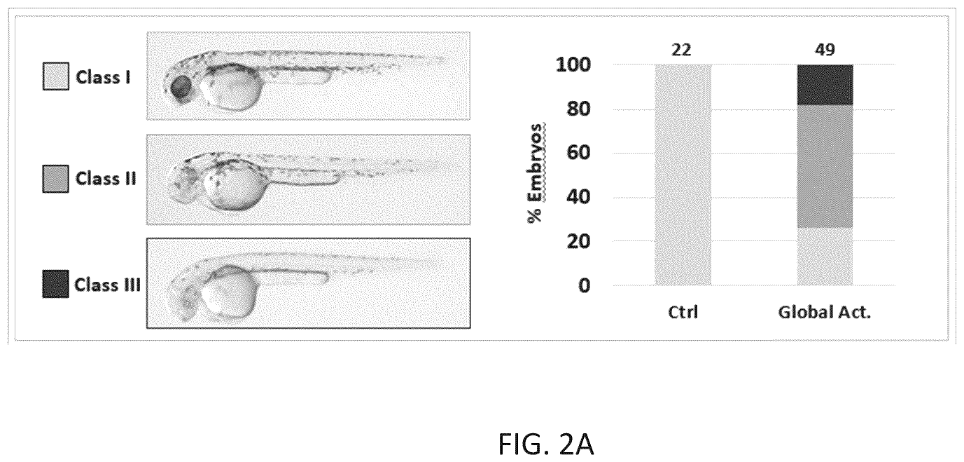

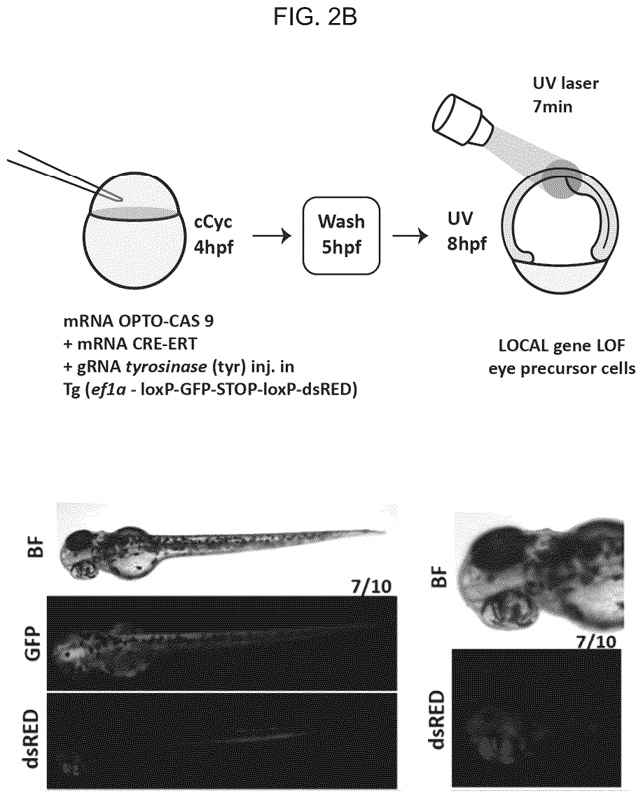

We choose to target tyrosinase (tyr) gene, since the phenotype is easy and quick to analyse in vivo and also because tyr is expressed in pigmented cells with different origin. Pigmented cells can originate from the RPE cell precursors (neuroepithelial derivative cells of the eyes) or from NCCs when they differenciate along the body axis. First, we evaluated the temporal response of OPTO-Cas9 to cCyc at global levels. We injected OPTO-Cas9 mRNA plus gTYR in wild-type zebrafish embryo incubated in cCyc at blastula stage (4 hpf). In whole embryos, activation of OPTO-Cas9 by uncaging cCyc for 4 minutes (2=365 nm at 75% epiboly, 8 hpf) leads to global reduction in pigmentation along the A/P axis (18.4% strong reduction, 55.1% partial reduction), as well as in the eyes (73.5% strong reduction), indicating that OPTO-Cas9 is effective in inactivating tyr both in RPE (retinal pigment epithelium) and in NCCs (neural crest cells) derivatives at the time of its activation (FIG. 2A).

We therefore asked whether OPTO-Cas9 can inactivate tyr with precise spatiotemporal control.

We injected OPTO-Cas9 mRNA plus gTYR plus CRE-ERT mRNA in a transgenic zebrafish Tg (ef1a: IoxP-GFP-STOP-IoxP-dsRED) that can switch from GFP (green fluorescence) to dsRED (red fluorescence) expression in a CRE-ERT dependent manner (FIG. 2B) (Sinha et al., 2010b. Since both CRE-ERT and OPTO-Cas9 can be activated by cCyc in a UV-dependent manner, we expected that cells expressing dsRED (mediated by CRE-ERT) would be depigmented by the parallel action of OPTO-Cas9 on tyr. We therefore illuminated presumptive RPE precursors cells to uncage cCyc, and thus activate CRE-ERT and OPTO-Cas9 in the same cells (FIG. 2B).

As expected, cells of the eye expressing dsRED also lack pigmentation, indicating that both CRE-ERT and OPTO-Cas9 were activated in these cells.

Strikingly, we can precisely modulate gene inactivation and pigmentation in only one eye, without effects on the contralateral dsRED negative eye (FIG. 2B), which is precluded by using promoter-specific mediating Cas9 expression (Di Donato et al., 2016; Hans et al., 2021).

Further, we asked whether OPTO-Cas9 can be important for analysing gene activity in vivo and thus for better understanding the precise spatiotemporal biological relevance of gene function, over a widely accepted demonstration of tyr inactivation in vivo.

Therefore, we sought to understand whether OPTO-Cas9 can offer a new dimension of biological analysis in vivo at single cell(s) resolution in the embryo.

### Example 3. Molecular Analysis of Vox Gene Inactivation by OPTO-Cas9

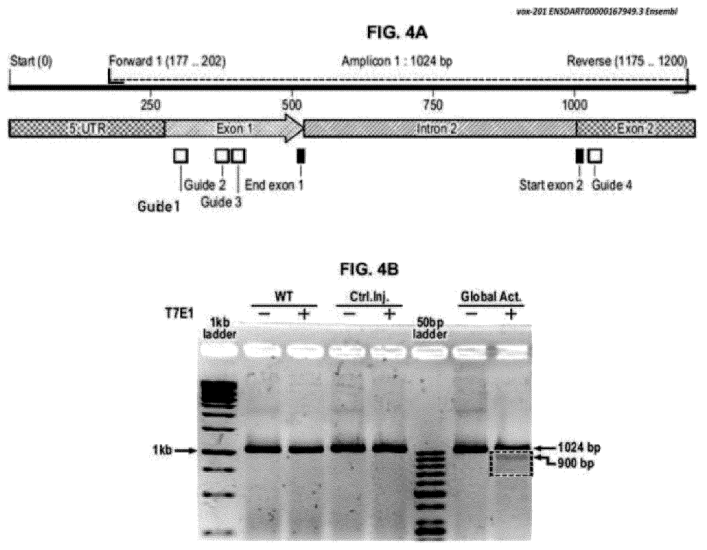

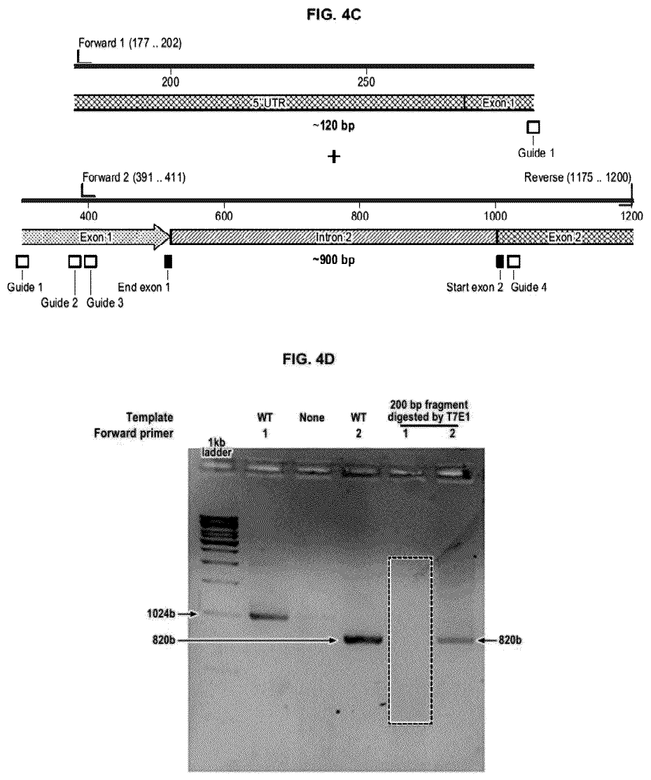

FIG. 4A shows a schematic representation of the vox gene (Ensemble vox-201 ENSDART00000167949.3) with the location of the four guides gVOX and two primers used to target and amplify vox gene, respectively.

The set of primers (forward 1 and reverse) generates a 1024 bp amplicon from gDNA. A T7 endonuclease assay was performed. T7 Endonuclease I (T7E1) recognizes and cleaves non-perfectly matched DNA. T7E1 assay reveals a 900 bp fragment in gDNA from globally activated zebrafish only, revealing that a mutation appeared in the vox gene under these conditions only.

These results demonstrate that the vox gene has been efficiently modified by OPTO-Cas9 after its activation.

Further, the sequencing of single embryo confirmed that mutations at vox sgRNA guide 1 locus were obtained, following OPTO-Cas9 photo-activation. High throughput sequencing and OPTO-Cas9 genotyping of individual embryos/larvae was performed following NGSelect Amplicon 2^{nd} PCR protocol (Eurofins Genomics). Complete indels count shows 6.6% recombination in one embryo, supporting the results of the PCR analysis performed after T7E1 assay (FIG. 4D).

### Example 4. Uses of OPTO-Cas9 for Precise Analyses on Embryonic Morphogenesis In Vivo

Until now, it has been difficult to perform spatially precise and fast gene inactivation for functional analyses of pleiotropic genes, thus overriding the effects of early temporal perturbations or spatial ectopic and undesired effects.

OPTO-Cas9 may allow to spatio-temporally decouple the activity of vox gene, which codes for a homeodomain transcription factor belonging to the VENTXINANOG family (Scerbo and Monsoro-Burq, 2020).

scRNA-sequencing (scRNA-seq) analyses show that vox is expressed in early pluripotent embryonic cells (Wagner et al., 2018), where vox interacts genetically and physically with pou5f3/oct4 for inhibiting precocious differentiation (Perez-Camps et al., 2016) whereas during epiboly and bud stages vox marks multipotent and undifferentiated NCCs as well as the multipotent neuro-mesodermal stem cells of the tailbud (Lukoseviciute et al., 2021) required for posterior axis elongation.

Therefore, scRNA-seq analyses suggest that vox, as marker of undifferentiated stem cells, may cover pleiotropic activities during embryonic development at different time points and in different cell types.

Shedding light on the biological relevance of a pleiotropic gene in a given tissue/cell types would be challenging and time-expensive with classical and new gene inactivation approaches (Hans et al., 2021).

To raise to that challenge, we injected OPTO-Cas9 mRNA plus gVOX (plus mRFP mRNA used as lineage tracer) in nacre zebrafish embryos, incubated with 6 μM cCyc from 4 hpf to 5 hpf (1 h of treatment).

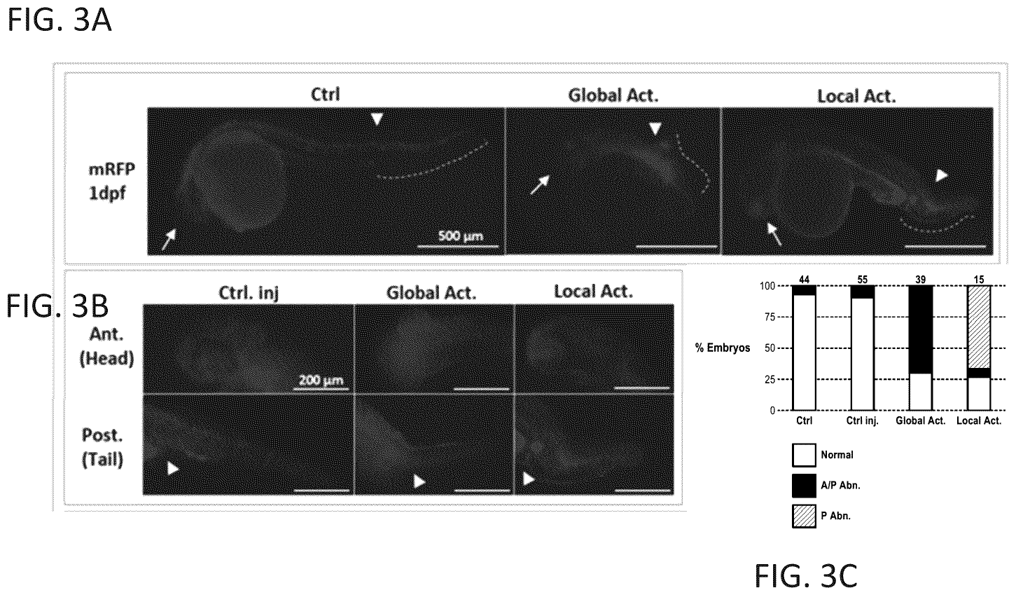

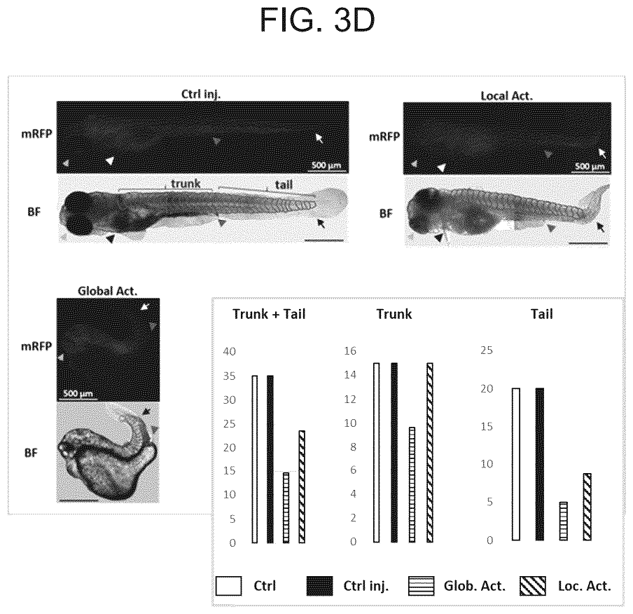

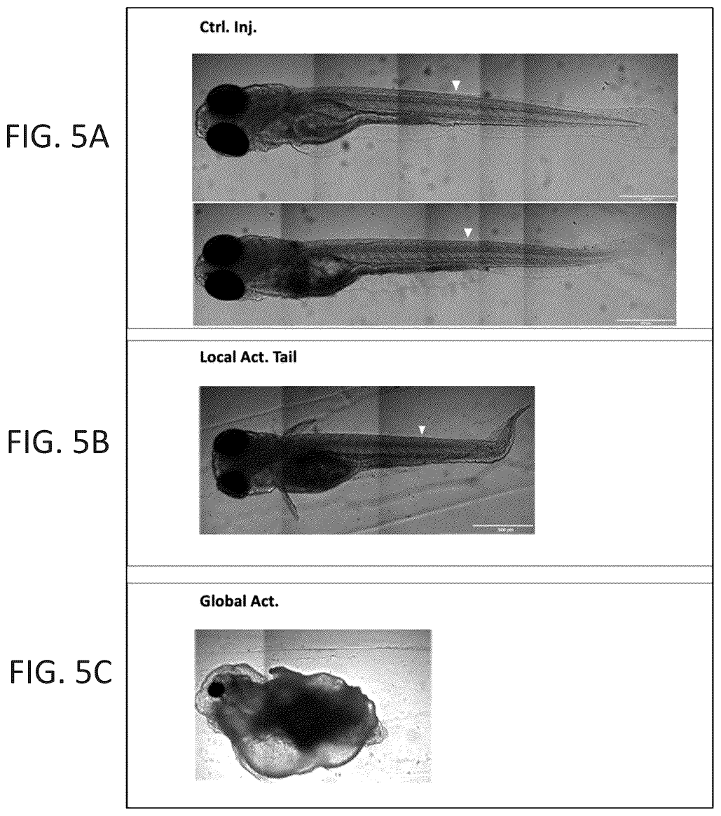

Global photoactivation of cCyc during epiboly (5-to-8 hpf, λ=365 nm for 4 minutes), induces severe morphological defects in embryo at 5 dpf (FIG. 3A, FIG. 5C).

Global inactivation of vox (FIG. 4A, 4B) strongly impairs head and eyes formation, the posterior axis fails to elongate and to form a correct trunk/tail structure. In contrast, control injected embryos did not display phenotypical or morphological defects (FIG. 3A, FIG. 5A). This confirmed the specificity of the process of the invention, without leakage. When we activated OPTO-Cas9 in a small group of presumptive tailbud cells in order to induce local and spatially confined vox LOF, we observed that only the posterior axis was impaired (FIG. 3A, 5B). These embryos share posterior axis defects with embryos in which OPTO-Cas9 was globally activated, but only global activation of OPTO-Cas9 and thus global vox LOF perturbated head and eye formation (FIG. 3B, FIG. 5C). Accordingly, morphology of tail somites was perturbed in both conditions of vox LOF (FIG. 3D, FIG. 5B).

At 3 dpf and 5 dpf, control injected embryos developed normally, whilst the anterior/posterior axis and the head of globally induced vox LOF zebrafish were strongly impaired (FIG. 3D, FIG. 5C).

In parallel, embryos in which vox activity was locally inactivated displayed only impaired posterior structures (mainly the tail), whereas the morphology of anterior structures (head and eyes) were normal (FIG. 3D, FIG. 5B).

We further quantified the mean of somite numbers (FIG. 3D). Control embryos (injected and non-injected) display a total of n=35 somites along the body axis (15 in the trunk, 20 in the tail). In contrast, global inactivation of vox strongly reduces somites number down to n=14 (9 in the trunk and 5 in the tail), whereas local vox inactivation reduces somites number to n=23 without effects on the trunk somites (n=15) but mainly on tail somites (n=8).

In conclusion, the chimeric protein OPTO-Cas9 can efficiently, quickly and precisely allow for phenotypical and morphological analyses of pleiotropic genes either at whole-organism resolution or in a small group of cells in vivo.

### Example 5. Use of a Chimeric Protein OPTO-Cas9-GR Allows Temporal Gene Inactivation in a Dexamethasone Dependent Manner

An OPTO-Cas9-GR has been generated with the glucocorticoid receptor. The present experiment illustrates the action of this chimeric protein after activation by addition of dexamethasone, on two different genes: tyr (as in example 2) and Vox (as in examples 3 and 4).

A) Zebrafish wild-type embryos were injected with OPTO-Cas9-GR mRNA together with gRNA against tyr at 1-cell stage and incubated with 10/μM of dexamethasone at 6 hpf for one hour. Zebrafish were rinsed and incubated in fresh embryo medium until phenotype analysis. Non-activated embryos were used as control (Ctrl, n=18).

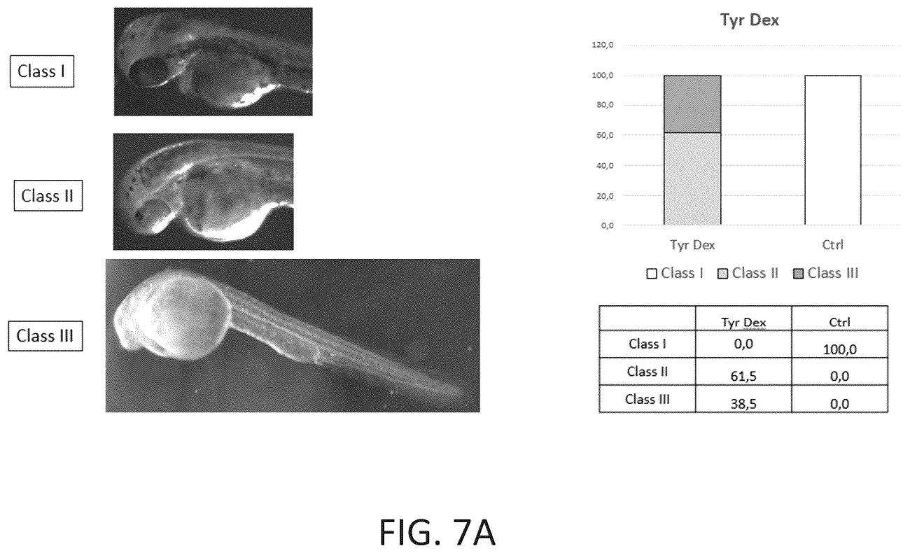

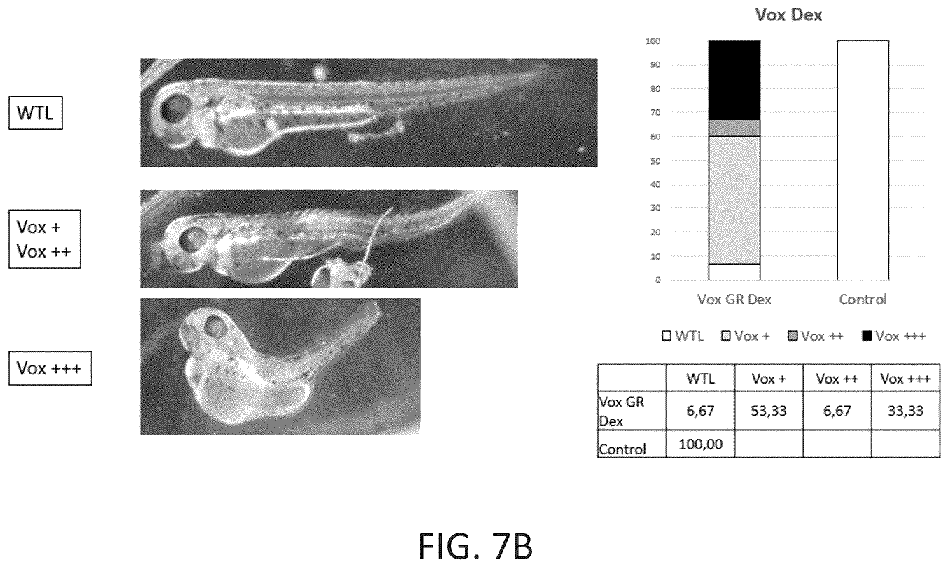

Pigmentation defects were analyzed in zebrafish at 2 dpf, in order to determine if the knock-out of the tyr gene, is effective. The results are illustrated in FIG. 7A.

Control embryos present a phenotype of Class I (white), wild type like phenotype, as expected.

Quantification of pigmentation defects indicates that no injected embryos (n=13) are of Class I, while 61.5% are of Class II (medium grey), i.e., partial pigmentation (in particular in the eyes), and 38.5% are of Class III (black), i.e., no pigmentation.

B) Zebrafish wild type embryos were injected with OPTO-Cas9-GR mRNA together with gRNA against vox at 1-cell stage and incubated with 10/μM of dexamethasone at 4 hpf (sphere stage) for one hour. Zebrafish were rinsed and incubated in fresh embryo medium until phenotype analysis.

Morphology of embryos was analyzed at 3 dpf and compared to non-activated embryos exhibiting wild type phenotype.

Compared to control embryos (white bar, n=5), global OPTO-Cas9-GR activation (n=15) reduces both trunk and tail somite number according to 3 levels of severity:
53.3% of embryos showed mild effect Vox+ (light grey, n=8),
About 6.7% medium effect Vox++ (medium grey, n=1) and
33.3% strong effect Vox+++ (black, n=5).

One embryo exhibited a wild type phenotype.

In conclusion, the chimeric protein OPTO-Cas9-GR can efficiently, quickly and precisely allow for phenotypical and morphological analyses of pleiotropic genes in vivo after addition of dexamethasone.

### REFERENCES IN ORDER OF CITATION IN THE TEXT

### Patents

WO 2013/158268

### Scientific Literature

Liu, K., Petree, C., Requena, T., Varshney, P. and Varshney, G. K. (2019). Expanding the CRISPR Toolbox in Zebrafish for Studying Development and Disease. Front Cell Dev Biol 7, 13.
Di Donato, V., De Santis, F., Auer, T. O., Testa, N., Sánchez-Iranzo, H., Mercader, N., Concordet, J. P. and Del Bene, F. (2016). 2C-Cas9: a versatile tool for clonal analysis of gene function. Genome Res 26, 681-692.
Alanis-Lobato, G., Zohren, J., McCarthy, A., Fogarty, N. M. E., Kubikova, N., Hardman, E., Greco, M., Wells, D., Turner, J. M. A. and Niakan, K. K. (2021). Frequent loss of heterozygosity in CRISPR-Cas9-edited early human embryos. Proc Natl Acad Sci USA 118, e2004832117.
Hemphill, J., Borchardt, E. K., Brown, K., Asokan, A. and Deiters, A. (2015). Optical Control of CRISPR/Cas9 Gene Editing. J Am Chem Soc 137, 5642-5645.
Nihongaki, Y., Kawano, F., Nakajima, T. and Sato, M. (2015b). Photoactivatable CRISPR-Cas9 for optogenetic genome editing. Nat Biotechnol 33, 755-760.
Polstein, L. R. and Gersbach, C. A. (2015). A light-inducible CRISPR-Cas9 system for control of endogenous gene activation. Nat Chem Biol 11, 198-200.
Feil, R., Brocard, J., Mascrez, B., LeMeur, M., Metzger, D. and Chambon, P. (1996). Ligand-activated site-specific recombination in mice. Proc Natl Acad Sci USA 93, 10887-10890.
Liu K I, Ramli M N, Woo C W, Wang Y, Zhao T, Zhang X, Yim G R, Chong B Y, Gowher A, Chua M Z, Jung J, Lee J H, Tan M H. A chemical-inducible CRISPR-Cas9 system for rapid control of genome editing. Nat Chem Biol. 2016 November; 12 (11): 980-987. doi: 10.1038/nchembio.2179. Epub 2016 Sep. 12. PMID: 27618190.
Sinha, D. K., Neveu, P., Gagey, N., Aujard, I., Benbrahim-Bouzidi, C., Le Saux, T., Rampon, C., Gauron, C., Goetz, B., Dubruille, S., et al. (2010a). Photocontrol of protein activity in cultured cells and zebrafish with one- and two-photon illumination. Chembiochem 11, 653-663.
Jinek, M., Chylinski, K., Fonfara, I., Hauer, M., Doudna, J. A. and Charpentier, E. (2012). A programmable dual-RNA-guided DNA endonuclease in adaptive bacterial immunity. Science 337, 816-821.
Hayashi K, Hashimoto K, Kusaka N, Yamazoe A, Fukaki H, Tasaka M, Nozaki H. Caged gene-inducer spatially and temporally controls gene expression and plant development in transgenic Arabidopsis plant. Bioorg Med Chem Lett. 2006 May 1; 16 (9): 2470-4. doi: 10.1016/j.bmcl.2006.01.103. Epub 2006 Feb. 9. PMID: 16480868.
Jao, L. E., Wente, S. R. and Chen, W. (2013). Efficient multiplex biallelic zebrafish genome editing using a CRISPR nuclease system. Proc Natl Acad Sci USA 110, 13904-13909.
Sinha, D. K., Neveu, P., Gagey, N., Aujard, I., Le Saux, T., Rampon, C., Gauron, C., Kawakami, K., Leucht, C., Bally-Cuif, L., et al. (2010b). Photoactivation of the CreER T2 recombinase for conditional site-specific recombination with high spatiotemporal resolution. Zebrafish 7, 199-204.
Hans, S., Zöller, D., Hammer, J., Stucke, J., Spieß, S., Kesavan, G., Kroehne, V., Eguiguren, J. S., Ezhkova, D., Petzold, A., et al. (2021). Cre-Controlled CRISPR mutagenesis provides fast and easy conditional gene inactivation in zebrafish. Nat Commun 12, 1125.
Scerbo, P. and Monsoro-Burq, A. H. (2020). The vertebrate-specific VENTX/NANOG gene empowers neural crest with ectomesenchyme potential. Sci Adv 6, eaaz1469.
Wagner, D. E., Weinreb, C., Collins, Z. M., Briggs, J. A., Megason, S. G. and Klein, A. M. (2018). Single-cell mapping of gene expression landscapes and lineage in the zebrafish embryo. Science 360, 981-987.
Perez-Camps, M., Tian, J., Chng, S. C., Sem, K. P., Sudhaharan, T., Teh, C., Wachsmuth, M., Korzh, V., Ahmed, S. and Reversade, B. (2016). Quantitative imaging reveals real-time Pou5f3-Nanog complexes driving dorsoventral mesendoderm patterning in zebrafish. Elife 5, e11475.
Lukoseviciute, M., Mayes, S. and Sauka-Spengler, T. (2021). Neuromesodermal progenitor origin of trunk neural crest in vivo. bioRxiv 2021.02.10.430513.

1. Chimeric protein comprising three covalently linked domains:
a RNA-guided DNA endonuclease;
a flexible peptide linker; and
a switchable receptor binding domain,
the endonuclease activity depending on the uncaging of a caged specific ligand of said switchable receptor binding domain.

2. Chimeric protein according to claim 1, wherein the RNA-guided DNA endonuclease is chosen among Cas9, Cas12a, Cas13a, Cas13b, and variants thereof.

3. Chimeric protein according to claim 1, wherein:
a) the switchable receptor binding domain is the hormone binding domain of the estrogen receptor (ERT) and the caged specific ligand is selected from caged cyclofen, caged 4-hydroxycyclofen, and caged tamoxifen, or
b) the switchable receptor binding domain is the hormone binding domain of the glucocorticoid receptor (GR) and the caged specific ligand is caged dexamethasone.

4. Chimeric protein according to claim 1, wherein the flexible peptide linker comprises between 17 and 27 amino acids, preferentially comprises 22 amino acids.

5. Chimeric protein according to claim 1, wherein the flexible peptide linker comprises at least one of the following domains:
a flexible linker having one glycine rich sequence, for example having a sequence GGGGS (SEQ ID NO. 1), and
a nuclear localization signal, for example having a sequence PKKKRKV (SEQ ID NO.2).

6. Chimeric protein according to claim 1, wherein the flexible peptide linker presents the sequence
SPVGGGGSRSPKKKRKVSPLEP (SEQ ID NO. 3).

7. A single-strand or double-strand polynucleotide encoding a chimeric protein according to claim 1.

8. A vector of expression adapted for a eukaryotic cell, comprising a double-strand polynucleotide of claim 7 under control of a suitable promoter.

9. A eukaryotic cell expressing, transiently or permanently, a chimeric protein according to claim 1.

10. A method for inducing nuclear translocation of the chimeric protein according to claim 1 in a eukaryotic cell, comprising:
providing a eukaryotic cell that contains said chimeric protein,
contacting said eukaryotic cell with a caged specific ligand of the switchable receptor binding domain,
irradiating the eukaryotic cell with a wavelength of light for a period of time sufficient to uncage the caged ligand, that will bind to the switchable receptor binding domain and induce the nuclear translocation of the chimeric protein.

11. A method for inducing the modification of an endogenous gene in a eukaryotic cell, comprising:
providing a eukaryotic cell that contains:
a chimeric protein according to claim 1, and
guide RNA(s) adapted to modify said endogenous gene,
contacting said eukaryotic cell with a caged specific ligand of the switchable receptor binding domain,
irradiating the eukaryotic cell with a wavelength of light for a period of time sufficient to uncage the caged ligand, that will bind to the switchable receptor binding domain and induce the nuclear translocation of the chimeric protein,
wherein once in the nucleus, the RNA-guided DNA endonuclease will act in conjunction with said guide RNA(s) for modifying said endogenous gene.

12. The method according to claim 10, wherein the caged ligand is chosen among the group consisting of: caged cyclofen, caged 4-hydroxycyclofen, caged dexamethasone, and caged tamoxifen.

13. The method according to claim 10, wherein the light is monophoton, preferably between 350 nm to 405 nm, or biphoton, preferably at 750 nm.

14. A kit for the implementation of the method according to claim 10, comprising:
a chimeric protein, or a single-strand polynucleotide, or a vector of expression;
a caged ligand, specifically binding to the switchable receptor binding domain of said chimeric protein under its uncaged form; and
optionally, light sources.

15. A kit for the implementation of the method according to claim 11, comprising:
a chimeric protein, or a single-strand polynucleotide, or a vector of expression;
guide RNA(s) adapted for modifying at least one specific gene;
a caged ligand, specifically binding to the switchable receptor binding domain of said chimeric protein under its uncaged form; and
optionally, light sources.
